(19) United States
(12) Patent Application Publication
(10) Pub. No.: US 2007/0225811 A1
(43) Pub. Date: Sep. 27, 2007

(54) Conformable orthopedic implant

(75) Inventors: Jeffrey L. Scifert, Arlington, TN (US); Scott D. Boden, Atlanta, GA (US)

Correspondence Address: LARSON NEWMAN ABEL POLANSKY & WHITE, LLP, 5914 WEST COURTYARD DRIVE, SUITE 200, AUSTIN, TX, 78730, US

(73) Assignee: SDGI HOLDINGS, INC., Wilmington, DE (US)

(21) Appl. No.: 11/386,460

(22) Filed: Mar. 22, 2006

(51) Int. Cl. A61F 2/44; A61F 2/30; A61F 2/28

(52) U.S. Cl. 623/17.14; 623/23.39; 623/23.51

(57) ABSTRACT

Compound orthopedic implants, intervertebral prosthetic implants and methods of treating a patient are provided. In an exemplary embodiment, a compound orthopedic implant comprises a first conformable body and a second conformable body overlying the first conformable body. The compound orthopedic implant can function as a conformable carrier for delivering a therapeutic agent to an orthopedic site.

### BACKGROUND

1. Field of the Disclosure

The present disclosure relates to the field of orthopedics and, more particularly, to conformable implants for treating void defects in bone.

2. Description of Related Art

In the field of orthopedics, it is often desirable to fill bony defects or voids and to deliver therapeutic agents to such sites. Such defects can be the direct result of disease or trauma, removal of diseased tissue or tumors, osteolytic conditions caused by wear debris from a prosthetic joint, or other degenerative or damaging conditions. Common sites that can present void defects include the cranium, fracture sites (especially compound fracture sites), areas comprising and proximate to synovial joints, and attachment sites for prosthetic joints.

A conventional treatment of the aforementioned conditions is compaction grafting, which involves compressing morselized cancellous allograft bone to fashion implants. Problems associated with compaction grafting include subsidence and the need to use synthetic “glues” such as polymethylmethacrylate. While cortical cancellous chips combined with metallic mesh and circlage wires have been used to fill voids in the acetabulum and proximal femur, cortical-cancellous chips handle poorly. The chips tend to behave like gravel and tend not to stay in a placement location unless enclosed by wire mesh or another retaining device. Furthermore, when methyl-methacrylate or like cement is pressurized in compaction grafting, large amounts of bone chips can become sequestered, therefore becoming biologically inactive. In addition to the aforementioned drawbacks, a recurring problem with compaction grafting is the significant number of smaller voids that often remain between a proximate surface of the implant and the proximate bone surface. The cumulative effect of these voids is often insufficient integration of the implant.

Revision and initial arthroplasty procedures can be especially problematic when sufficient osteo-integration does not occur. Prosthetic joints are typically formed of substantially rigid metals, alloys, polymers, or polymer blends in order to provide a structure that can withstand the loading presented in the joint regions. Biocompatibility and bioresorbability behavior of a material are also significant criteria for a successful implant, thereby reducing the number of available materials. Wear occurring at the interface of surfaces within the prosthetic joint can be a significant contributor to joint failure as well as to deleterious effects in collateral systems resulting from wear debris. For example, wear debris can contribute to osteolysis in surrounding bones, including the prosthetic implant recipient sites, thereby making revision surgery necessary and, at the same time, adversely affecting the chance of success of the revision surgery using conventional techniques.

### SUMMARY

Accordingly, the present disclosure is directed to various embodiments of a compound orthopedic implant, an intervertebral prosthetic implant and a method of treating a patient. In an exemplary embodiment, a compound orthopedic implant includes a first conformable body and a second conformable body overlying the first conformable body.

In another exemplary embodiment, a compound orthopedic implant includes a first conformable body comprising ceramic particles and collagen and a second conformable body overlying the first conformable body. The second conformable body also is comprised of ceramic particles and collagen. At least one of the first conformable body or the second conformable body includes a therapeutic agent.

In another exemplary embodiment, a method of treating a patient includes the steps of determining an orthopedic characteristic of the patient and configuring a compound orthopedic implant based on the orthopedic characteristic. The compound orthopedic implant includes a first conformable body and a second conformable body. The compound orthopedic implant is delivered to a point of use at an orthopedic site.

In another exemplary embodiment, a kit for field use includes a first conformable body, a second conformable body, and instructions for utilizing the first and second conformable bodies as a compound orthopedic implant.

In another exemplary embodiment, an intervertebral prosthetic implant includes a substantially rigid first member having an engagement surface configured to engage a first vertebra. A first conformable body is disposed on the engagement surface of the first member. A second conformable body is disposed on the first conformable body.

### BRIEF DESCRIPTION OF THE DRAWINGS

The present disclosure may be better understood, and its numerous features and advantages made apparent to those skilled in the art by referencing the accompanying drawings.

FIG. 1 is a top plan view of an embodiment of a compound orthopedic implant;

FIG. 2 is a lateral cross-sectional view along line 2-A of FIG. 1;

FIG. 3 is a lateral view of a portion of a vertebral column;

FIG. 4 is a lateral view of a pair of adjacent vertrebrae;

FIG. 5 is a top plan view of a vertebra;

FIG. 6 is an anterior view of a first embodiment of an intervertebral prosthetic disc;

FIG. 7 is an exploded anterior view of the first embodiment of the intervertebral prosthetic disc;

FIG. 8 is a lateral view of the first embodiment of the intervertebral prosthetic disc;

FIG. 9 is an exploded lateral view of the first embodiment of the intervertebral prosthetic disc;

FIG. 10 is a plan view of a superior half of the first embodiment of the intervertebral prosthetic disc;

FIG. 11 is another plan view of the superior half of the first embodiment of the intervertebral prosthetic disc;

FIG. 12 is a plan view of an inferior half of the first embodiment of the intervertebral prosthetic disc;

FIG. 13 is a plan view of an inferior half of the first embodiment of the intervertebral prosthetic disc;

FIG. 14 is an exploded lateral view of the first embodiment of the intervertebral prosthetic disc installed within an intervertebral space between a pair of adjacent vertrebrae; and

FIG. 15 is an anterior view of the first embodiment of the intervertebral prosthetic disc installed within an intervertebral space between a pair of adjacent vertrebrae.

The use of the same reference symbols in different drawings indicates similar or identical items.

### DETAILED DESCRIPTION

The teachings of the present application can find utility in various orthopedic situations, such as, e.g., fracture repair, prosthetic implants for total and partial joint replacement (e.g., knee, hip, shoulder or spinal), cranium repair, as well as adjuncts in various orthopedic surgical procedures or the like.

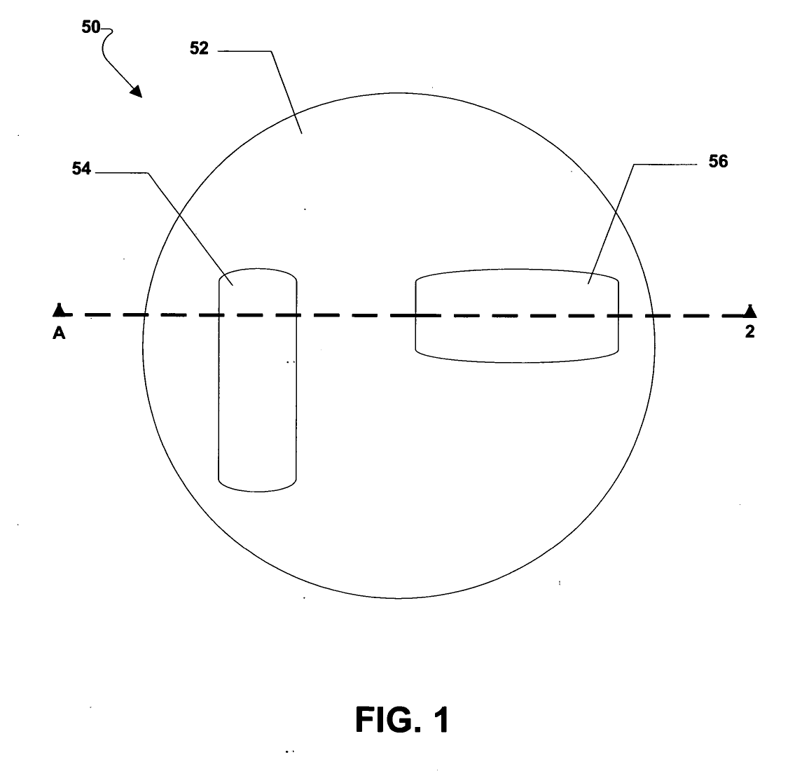

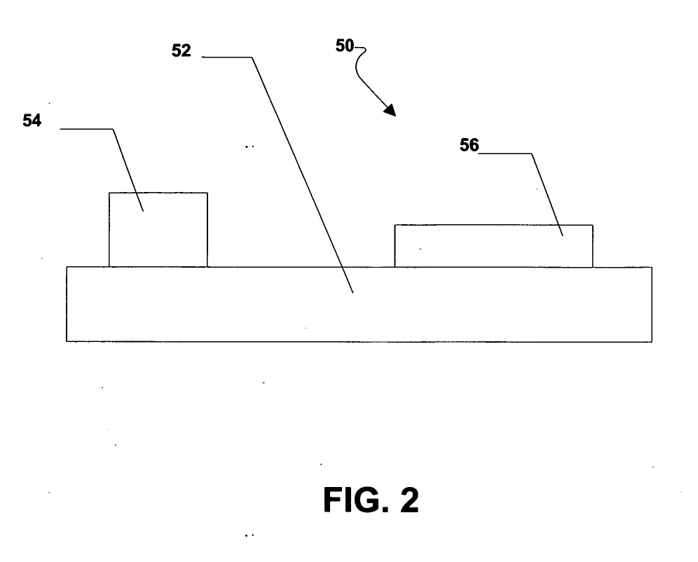

With reference to FIGS. 1 and 2, in various embodiments, a compound orthopedic implant 50 includes a first conformable body 52 and a second conformable body 54 overlying the first conformable body 52. Each conformable body can be formed of ceramic particles in a carrier. The ceramic particles can be selected from those that interact favorably with the human biologic system and that may promote bone growth. Exemplary suitable ceramic materials include hydroxyapatite (HA), hydroxyapatite tricalcium phosphate (HATCP), calcium phosphate, calcium sulfate, or any combination thereof.

The carrier can include a collagen material, such as fibrous collagen. The fibrous collagen can be utilized in its non-gelatinized state. In certain embodiments, the collagen is at least partially crosslinked to obtain desired mechanical properties in the conformable body. The collagen can be crosslinked using art-recognized methods. The choice of crosslinking method can depend in part on the extent of crosslinking desired, other manufacturing parameters, and/or the identity of other constituents or additives in the conformable body. For example, crosslinking can be effected by exposure to a radiation source, such as an ultraviolet radiation source, an infrared source, a gamma-radiation source, an e-beam source, or any combination thereof. In other examples, crosslinking can be effected by thermal treatment or by chemical treatment. In various exemplary embodiments, these treatments can result in crosslinking of the bulk material of the conformable body or only a portion of the bulk material. When crosslinking is effected in a portion of the bulk material of the body, the bulk material in regions proximate to the primary crosslinked portion can be crosslinked to a lesser extent, resulting in a gradient of extent of crosslinking in the bulk material. Partial crosslinking can be carried out using a number of conventional methods. For example, when crosslinking is carried out by irradiation, portions of the conformable body can be masked to minimize exposure to the energy. These partially crosslinked embodiments can be used when it is determined that the presence of certain strength properties is more desirable than a high degree of conformability in certain portions of the body.

Certain embodiments can include generally biocompatible polymers, such as a polyurethane material, a polyolefin material, a polystyrene, a polyurea, a polyamide, a polyaryletherketone (PAEK) material, a silicone material, a hydrogel material, or any alloy, blend or copolymer thereof. An exemplary polyolefin material can include polypropylene, polyethylene, halogenated polyolefin, fluoropolyolefin, polybutadiene, or any combination thereof. An exemplary polyaryletherketone (PAEK) material can include polyetherketone (PEK), polyetheretherketone (PEEK), polyetherketoneketone (PEKK), polyetherketoneetherketoneketone (PEKEKK), or any combination thereof. An exemplary silicone can include dialkyl silicones, fluorosilicones, or any combination thereof. An exemplary hydrogel can include polyacrylamide (PAAM), poly-N-isopropylacrylamine (PNIPAM), polyvinyl methylether (PVM), polyvinyl alcohol (PVA), polyethyl hydroxyethyl cellulose, poly (2-ethyl) oxazoline, polyethyleneoxide (PEO), polyethylglycol (PEG), polyacrylacid (PAA), polyacrylonitrile (PAN), polyvinylacrylate (PVA), polyvinylpyrrolidone (PVP), or any combination thereof.

In various exemplary embodiments, the polymer material(s) of one or more of the conformable bodies can be crosslinked. In one exemplary embodiment, the bulk polymeric material is crosslinkable using radiation. The bulk polymeric material can include a photoinitiator or a photosensitizer. In another exemplary embodiment, the bulk polymeric material is thermally crosslinkable and includes a heat activated catalyst. Further, the bulk polymeric material can include a crosslinking agent, which can act to form crosslinks between polymer chains.

For example, for polyurethane materials, a suitable chemical crosslinking agent can include low molecular weight polyols or polyamines. An example of such a suitable chemical crosslinking agent can include trimethylolpropane, pentaerythritol, ISONOL® 93 curative from Dow Chemical Co., trimethylolethane, triethanolamine, Jeffamines, 1,4-butanediamine, xylene diamine, diethylenetriamine, methylene dianiline, diethanolamine, or any combination thereof.

For silicone materials, a suitable chemical crosslinking agent can include tetramethoxysilane, tetraethoxysilane, tetrapropoxysilane, tetrabutoxysilane, methyltrimethoxysilane, methyltriethoxysilane, vinyltrimethoxysilane, vinyltriethoxysilane, phenyltrimethoxysilane, phenyltriethoxysilane, 3-cyanopropyltrimethoxysilane, 3-cyanopropyltriethoxysilane, 3-(glycidyloxy)propyltriethoxysilane, 1,2-bis(trimethoxysilyl)ethane, 1,2-bis(triethoxysilyl)ethane, hexaethoxydisiloxane, or any combination thereof.

Additionally, for polyolefin materials, a suitable chemical crosslinking agent can include an isocyanate, a polyol, a polyamine, or any combination thereof. The isocyanate can include 4,4′-diphenylmethane diisocyanate, polymeric 4,4′-diphenylmethane diisocyanate, carbodiimide-modified liquid 4,4′-diphenylmethane diisocyanate, 4,4′-dicyclohexylmethane diisocyanate, p-phenylene diisocyanate, toluene diisocyanate, isophoronediisocyanate, p-methylxylene diisocyanate, m-methylxylene diisocyanate, o-methylxylene diisocyanate, or any combination thereof. The polyol can include polyether polyol, hydroxy-terminated polybutadiene, polyester polyol, polycaprolactone polyol, polycarbonate polyol, or any combination thereof. Further, the polyamine can include 3,5-dimethylthio-2,4-toluenediamine or one or more isomers thereof; 3,5-diethyltoluene-2,4-diamine or one or more isomers thereof; 4,4′-bis-(sec-butylamino)-diphenylmethane; 1,4-bis-(sec-butylamino)-benzene, 4,4′-methylene-bis-(2-chloroaniline); 4,4′-methylene-bis-(3-chloro-2,6-diethylaniline); trimethylene glycol-di-p-aminobenzoate; polytetramethyleneoxide-di-p-aminobenzoate; N,N′-dialkyldiamino diphenyl methane; p,p′-methylene dianiline; phenylenediamine; 4,4′-methylene-bis-(2-chloroaniline); 4,4′-methylene-bis-(2,6-diethylaniline); 4,4′-diamino-3,3′-diethyl-5,5′-dimethyl-diphenylmethane; 2,2′,3,3′-tetrachloro diamino diphenylmethane; 4,4′-methylene-bis-(3-chloro-2,6-diethylaniline); or any combination thereof.

In another embodiment, the chemical crosslinking agent is a polyol curing agent. The polyol curing agent can include ethylene glycol; diethylene glycol; polyethylene glycol; propylene glycol; polypropylene glycol; lower molecular weight polytetramethylene ether glycol; 1,3-bis(2-hydroxyethoxy) benzene; 1,3-bis-[2-(2-hydroxyethoxy)ethoxy]benzene; 1,3-bis-{2-[2-(2-hydroxyethoxy)ethoxy]ethoxy}benzene; 1,4-butanediol; 1,5-pentanediol; 1,6-hexanediol; resorcinol-di-(β-hydroxyethyl) ether; hydroquinone-di-(β-hydroxyethyl) ether; trimethylol propane, and any mixtures thereof.

One or more of the conformable bodies can be coated with, embedded with or otherwise include a therapeutic agent, such as a biological factor that can promote bone on-growth or bone in-growth. For example, the therapeutic agent can include bone morphogenetic protein (BMP), cartilage-derived morphogenetic protein (CDMP), platelet derived growth factor (PDGF), insulin-like growth factor (IGF), LIM mineralization protein, fibroblast growth factor (FGF), osteoblast growth factor, vascular growth factors, TGFβ, stem cells, combinations thereof, or any material considered to be beneficial in the filling of bone or cartilaginous voids and the remodeling thereof into solid, healthy bone or cartilage through the processes of osteointegration (including, e.g., osteogenesis, osteoinduction and osteoconduction). Further, the stem cells can include bone marrow-derived stem cells, lipo-derived stem cells, or a combination thereof.

A number of implant configurations are possible based on clinical need. For example, as depicted in FIGS. 1 & 2, when additional thickness or therapeutic agent delivery is needed in a discrete region of the implant, the second conformable body 54 may only partially overlie the first conformable body 52—i.e., overlying only in the region of interest. In other embodiments (not shown), the second conformable body can completely overlie the first conformable body. Alternatively, the implant can further include a third conformable body 56 overlying the first conformable body 52. In this three-component configuration, the second conformable body 54 can overlie a first portion of the first conformable body 52 and the third conformable body 56 can overlie a second portion of the first conformable body 52. The third conformable body can partially or fully overlie the second conformable body. Alternatively, the third conformable body can partially or fully overlie both the first and second conformable bodies. Alternatively, the first and second portions of the first conformable body may not overlap each other and, further, the first and second portions may not be contiguous. Various other alternative embodiments can include additional conformable bodies as necessary. The adaptability of the present compound implant allows for nearly immediate adaptability during surgery to address regions of excessive bone loss and the like. For example, it may not be practical or effective to utilize a preformed, non-adaptable implant for treating a void produced by surgical removal of diseased bone or tumor removal. However, the present compound implant can find utility in such circumstances.

The adaptable configuration of the present compound implant can also allow for selective or time released delivery of one or more therapeutic agents. In certain embodiments, at least one of the conformable bodies is coated with, embedded with or otherwise includes a therapeutic agent, as identified supra. The therapeutic agent can be introduced during manufacture or post-manufacture, such as by surgical staff before implantation. In certain embodiments, all of the conformable bodies can include a therapeutic agent. In alternative two-component configurations, the first conformable body can include a therapeutic agent and the second conformable body can be substantially free of therapeutic agents. In embodiments having three or more conformable bodies, one or more of the conformable bodies can be substantially free of therapeutic agents, while one or more of the bodies can include a therapeutic agent. This aspect of the invention not only allows for targeted and concentrated delivery, but also for utilization of therapeutic agents that may be particularly scarce.

In various embodiments of the present compound implants, the weight ratio of ceramic particles to collagen in all of the conformable bodies is between about 5:1 and about 20:1. In alternative embodiments, at least one of the conformable bodies has a weight ratio of ceramic particles to collagen greater than the weight ratio of ceramic particles to collagen in another of the conformable bodies. In certain embodiments, the conformable bodies with the greater weight ratio have a weight ratio of ceramic particles to collagen of between about 22:1 and 40:1. Furthermore, in various embodiments, the extent of crosslinking of collagen and/or polymer can differ between or among the conformable bodies. Since the relative amount of collagen and the extent of crosslinking can affect the mechanical properties (e.g., rigidity) of the material, the above-described characteristics can allow for tailoring of the conformability of the compound implant. In addition, depending on the identity, form and amount of therapeutic agent, these characteristics can affect the availability of therapeutic agent at the implant site; therefore, a timed release of therapeutic agent may be provided, if indicated.

In addition to compositional adaptability, each of the conformable bodies can be provided in various shapes and dimensions, such as various thicknesses, in order to allow significant adaptability in the field. The conformable bodies can be produced using art-recognized forming techniques such as molding, injection molding, slip casting or the like. With certain embodiments, it may be beneficial to avoid excessive heat exposure in order to maintain the therapeutic effect of a therapeutic agent, to avoid unwanted crosslinking or to maintain the structural integrity of the collagen matrix.

The present compound implant can be provided in kit form, including multiple conformable bodies and instructions for stacking or otherwise utilizing the conformable bodies as a compound orthopedic implant. One or more of the conformable bodies can be preloaded with a suitable therapeutic agent. In addition or alternatively, the kit can include a discrete supply of a therapeutic agent for use in the implant. The instructions can direct a user to embed the kit-supplied therapeutic agent in at least one of the conformable bodies and/or to embed a separately obtained therapeutic agent in a conformable body.

Certain embodiments of the conformable bodies can be pressed together utilizing finger pressure, so that the collagen fibers in one body physically integrate with those in the adjoining body. Further, biocompatible chemical bonding agents can be utilized if desired. One or more of the conformable bodies can be substantially preformed before implantation, for example, by press forming around a prosthetic member before implantation.

For example, a kit for use with revision surgery for total hip replacements can include a hemispherical or conical first conformable body for use behind the acetabular cup, where osteolytic lesions can typically occur due to wear debris induced osteolysis. Because of the relatively stable shape of the prosthetic hip implant, the shaped first conformable body can be made less conformable material, via compositional selection and/or increased crosslinking, as discussed above. Irregularly shaped voids can be addressed with additional conformable bodies made of more conformable material. A therapeutic material, such as BMP can be “preloaded” into the crosslinked, shaped body and BMP can be supplied in the kit for embedding into one or more of the additional conformable bodies.

Another aspect of the present disclosure is directed to a method of treating a patient comprising the steps of determining an orthopedic characteristic of the patient; configuring a compound orthopedic implant based on the orthopedic characteristic; and delivering the compound orthopedic implant to an orthopedic site. The compound orthopedic implant can include multiple conformable bodies of varying compositions as described previously herein. The method can include embedding or otherwise introducing a therapeutic agent into or on at least one of the conformable bodies. The method can also include placing one of the conformable bodies on another of the conformable bodies in a stacked arrangement.

Another aspect of the present compound implant is its use as a compound conformable adjunct on an otherwise rigid prosthetic implant (i.e., a substrate). The conformable body can be affixed to, attached to, or otherwise deposited on, an engagement surface of the substrate. The conformable body can be chemically bonded to the substrate, e.g., using an adhesive or another chemical bonding agent. Further, the conformable body can be mechanically anchored to the substrate using a mechanical fastener.

Before the conformable body is deposited, or otherwise affixed to the substrate, the substrate's engagement surface can be modified to promote adhesion of the conformable body to the engagement surface. For example, the engagement surface can be roughened to promote adhesion of the conformable body. For example, the roughening process can include acid etching; knurling; application of a bead coating, e.g., cobalt chrome beads; application of a roughening spray; e.g., titanium plasma spray (TPS); laser blasting; or any other similar process or method.

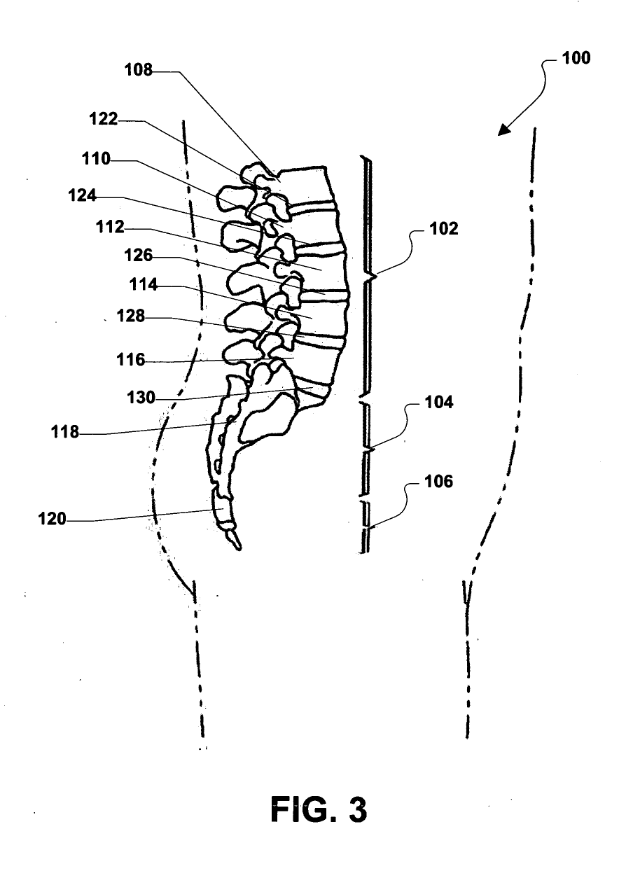

An exemplary application of the compound conformable adjunct is in combination with an intervertebral prosthetic disc. With particular reference to intervertebral embodiments, FIG. 3 shows a portion of a vertebral column, designated 100. As depicted, the vertebral column 100 includes a lumbar region 102, a sacral region 104, and a coccygeal region 106. As is known in the art, the vertebral column 100 also includes a cervical region and a thoracic region. For clarity and ease of discussion, the cervical region and the thoracic region are not illustrated.

As shown in FIG. 3, the lumbar region 102 includes a first lumbar vertebra 108, a second lumbar vertebra 110, a third lumbar vertebra 112, a fourth lumbar vertebra 114, and a fifth lumbar vertebra 116. The sacral region 104 includes a sacrum 118. Further, the coccygeal region 106 includes a coccyx 120.

As depicted in FIG. 3, a first intervertebral lumbar disc 122 is disposed between the first lumbar vertebra 108 and the second lumbar vertebra 110. A second intervertebral lumbar disc 124 is disposed between the second lumbar vertebra 110 and the third lumbar vertebra 112. A third intervertebral lumbar disc 126 is disposed between the third lumbar vertebra 112 and the fourth lumbar vertebra 114. Further, a fourth intervertebral lumbar disc 128 is disposed between the fourth lumbar vertebra 114 and the fifth lumbar vertebra 116. Additionally, a fifth intervertebral lumbar disc 130 is disposed between the fifth lumbar vertebra 116 and the sacrum 118.

In a particular embodiment, if one of the intervertebral lumbar discs 122, 124, 126, 128, 130 is diseased, degenerated, damaged, or otherwise in need of replacement, that intervertebral lumbar disc 122, 124, 126, 128, 130 can be at least partially removed and replaced with an intervertebral prosthetic disc according to one or more of the embodiments described herein. In a particular embodiment, a portion of the intervertebral lumbar disc 122, 124, 126, 128, 130 can be removed via a discectomy, or a similar surgical procedure, well known in the art. Further, removal of intervertebral lumbar disc material can result in the formation of an intervertebral space (not shown) between two adjacent lumbar vertebrae.

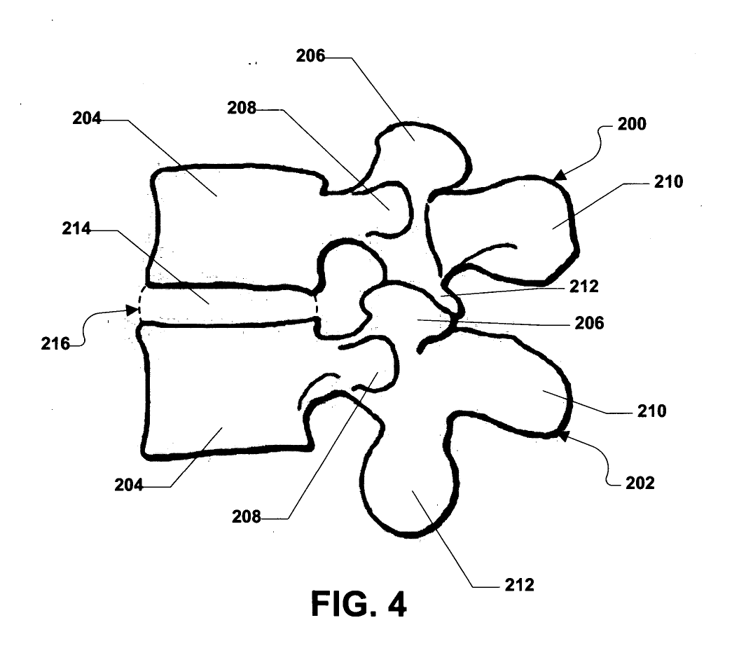

FIG. 4 depicts a detailed lateral view of two adjacent vertebrae, e.g., two of the lumbar vertebra 108, 110, 112, 114, 116 shown in FIG. 1. FIG. 2 illustrates a superior vertebra 200 and an inferior vertebra 202. As shown, each vertebra 200, 202 includes a vertebral body 204, a superior articular process 206, a transverse process 208, a spinous process 210 and an inferior articular process 212. FIG. 4 further depicts an intervertebral space 214 that can be established between the superior vertebra 200 and the inferior vertebra 202 by removing an intervertebral disc 216 (shown in dashed lines). As described in greater detail below, an intervertebral prosthetic disc according to one or more of the embodiments described herein can be installed within the intervertebral space 212 between the superior vertebra 200 and the inferior vertebra 202.

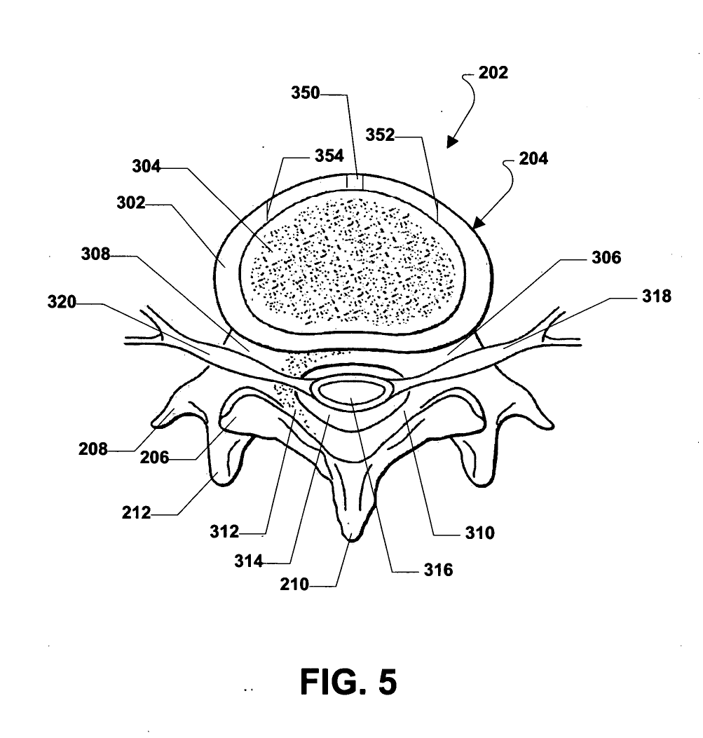

Referring to FIG. 5, a vertebra, e.g., the inferior vertebra 202 (FIG. 4), is illustrated. As shown, the vertebral body 204 of the inferior vertebra 202 includes a cortical rim 302 composed of cortical bone. Also, the vertebral body 204 includes cancellous bone 304 within the cortical rim 302. The cortical rim 302 is often referred to as the apophyseal rim or apophyseal ring. Further, the cancellous bone 304 is softer than the cortical bone of the cortical rim 302.

As illustrated in FIG. 5, the inferior vertebra 202 further includes a first pedicle 306, a second pedicle 308, a first lamina 310, and a second lamina 312. Further, a vertebral foramen 314 is established within the inferior vertebra 202. A spinal cord 316 passes through the vertebral foramen 314. Moreover, a first nerve root 318 and a second nerve root 320 extend from the spinal cord 316.

The vertebrae that make up the vertebral column have slightly different appearances as they range from the cervical region to the lumbar region of the vertebral column. However, all of the vertebrae, except the first and second cervical vertebrae, have the same basic structures, e.g., those structures described above in conjunction with FIG. 4 and FIG. 5. The first and second cervical vertebrae are structurally different than the rest of the vertebrae in order to support a skull.

FIG. 5 further depicts a keel groove 350 that can be established within the cortical rim 302 of the inferior vertebra 202. Further, a first corner cut 352 and a second corner cut 354 can be established within the cortical rim 302 of the inferior vertebra 202. In a particular embodiment, the keel groove 350 and the corner cuts 352, 354 can be established during surgery to install an intervertebral prosthetic disc according to one or more of the embodiments described herein. The keel groove 350 can be established using a keel-cutting device, e.g., a keel chisel designed to cut a groove in a vertebra, prior to the installation of the intervertebral prosthetic disc. Further, the keel groove 350 is sized and shaped to receive and engage a keel, described below, that extends from an intervertebral prosthetic disc according to one or more of the embodiments described herein. The keel groove 350 can cooperate with a keel to facilitate proper alignment of an intervertebral prosthetic disc within an intervertebral space between an inferior vertebra and a superior vertebra.

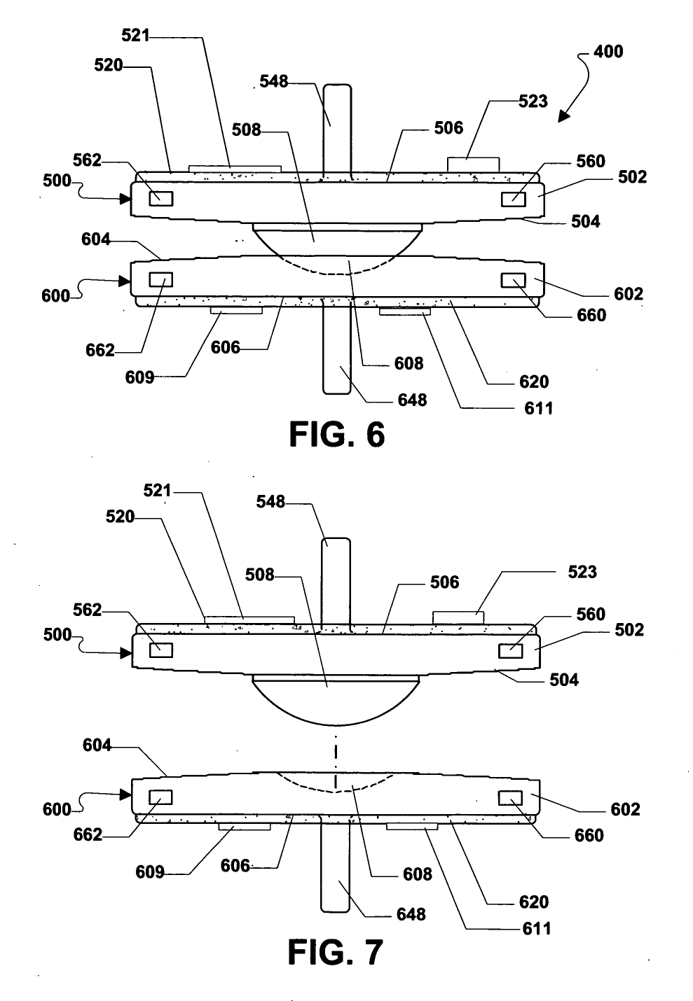

As shown in FIGS. 6-15, another aspect is directed to an intervertebral prosthetic implant generally designated as 400. As illustrated, the intervertebral prosthetic implant 400 includes a substantially rigid first member (configured as a superior component in this embodiment) 500 and a substantially rigid second member (configured as an inferior component) 600. In a particular embodiment, the components 500, 600 can be made from one or more extended use biocompatible materials. For example, the materials can be metal materials, ceramic materials, polymer materials, or composite materials that include metals, polymers, ceramics or combinations thereof.

The metals can be pure metals or metal alloys. The pure metals can include titanium. Moreover, the metal alloys can include stainless steel, a cobalt-chrome-molybdenum alloy, e.g., ASTM F-999 or ASTM F-75, a titanium alloy, or a combination thereof.

The polymer materials can include polyurethane materials, polyolefin materials, polyether materials, silicone materials, hydrogel materials, or a combination thereof. Further, the polyolefin materials can include polypropylene, polyethylene, halogenated polyolefin, fluoropolyolefin, or a combination thereof. The polyether materials can include polyetherketone (PEK), polyetheretherketone (PEEK), polyetherketoneketone (PEKK), polyaryletherketone (PAEK), or a combination thereof. Alternatively, the components 500, 600 can be made from any other substantially rigid biocompatible materials.

In a particular embodiment, the superior component 500 includes a superior support plate 502 that has a superior articular surface 504 and a superior engagement surface 506. In a particular embodiment, the superior articular surface 504 can be generally curved and the superior engagement surface 506 can be substantially flat. In an alternative embodiment, the superior articular surface 504 can be substantially flat and at least a portion of the superior engagement surface 506 can be generally curved.

As illustrated in FIG. 6 through FIG. 9, an articulation member 508 having an articulation surface extends from the superior articular surface 504 of the superior support plate 502. In a particular embodiment, the articulation member 508 has a hemi-spherical shape. Alternatively, the articulation member 508 can have an elliptical shape, a cylindrical shape, or other arcuate shape. Moreover, the articulation member 508 can be formed with a groove 510. Although the articulation member is depicted as forming a substantially monolithic structure with the superior component, the articulation member can be a separable, discrete nucleus with multiple articulation surfaces for engaging both the superior and inferior components.

As further illustrated, the superior component 500 includes a first conformable body 520 that can be affixed to, attached to, or otherwise disposed on, the superior engagement surface 506. The first conformable body 520 can be chemically or mechanically attached as described previously herein. The superior component can also include a second conformable body 521 and a third conformable body 523, both overlying the first conformable body 520. Each of the conformable bodies can be formulated as described supra. Further, one or more of the conformable bodies can include a therapeutic agent as described supra.

FIG. 6 through FIG. 9 show that the superior component 500 can include a superior keel 548 that extends from superior engagement surface 506. During installation, described below, the superior keel 548 can at least partially engage a keel groove that can be established within a cortical rim of a vertebra.

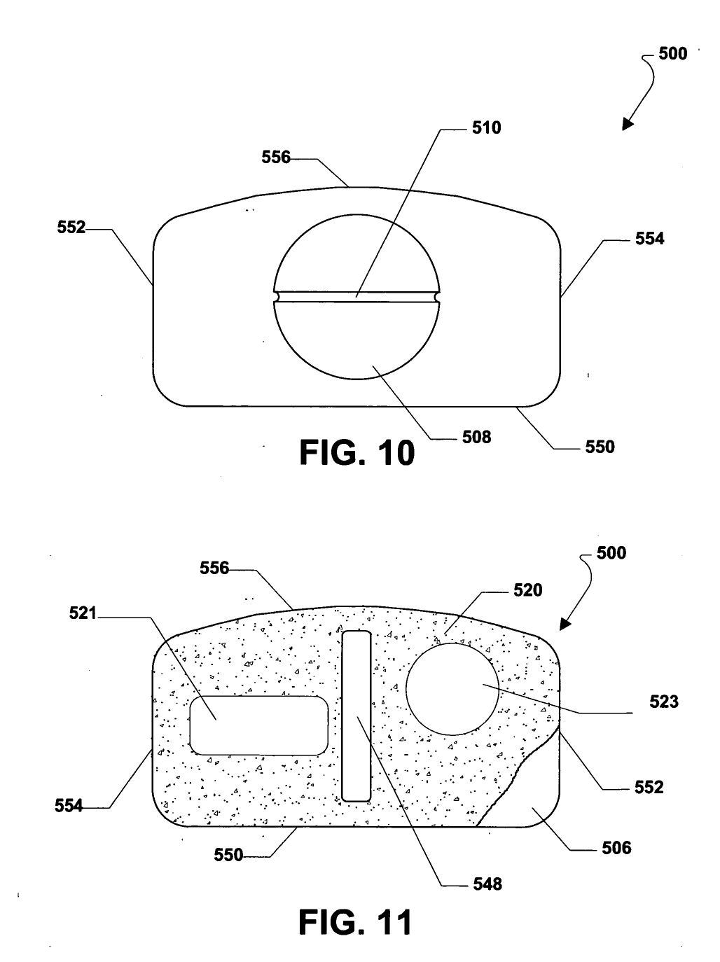

As illustrated in FIG. 10 and FIG. 11, the superior component 500 can be generally rectangular in shape. For example, the superior component 500 can have a substantially straight posterior side 550. A first straight lateral side 552 and a second substantially straight lateral side 554 can extend substantially perpendicular from the posterior side 550 to an anterior side 556. In a particular embodiment, the anterior side 556 can curve outward such that the superior component 500 is wider through the middle than along the lateral sides 552, 554. Further, in a particular embodiment, the lateral sides 552, 554 are substantially the same length.

FIG. 6 and FIG. 7 show that the superior component 500 includes a first implant inserter engagement hole 560 and a second implant inserter engagement hole 562. In a particular embodiment, the implant inserter engagement holes 560, 562 are configured to receive respective dowels, or pins, that extend from an implant inserter (not shown) that can be used to facilitate the proper installation of an intervertebral prosthetic implant, e.g., the intervertebral prosthetic implant 400 shown in FIG. 6 through FIG. 13.

In a particular embodiment, the inferior component 600 includes an inferior support plate 602 that has an inferior articular surface 604 and an inferior engagement surface 606. In a particular embodiment, the inferior articular surface 604 can be generally curved and the inferior engagement surface 606 can be substantially flat. In an alternative embodiment, the inferior articular surface 604 can be substantially flat and at least a portion of the inferior engagement surface 606 can be generally curved.

As illustrated in FIG. 6 through FIG. 9, a depression 608 extends into the inferior articular surface 604 of the inferior support plate 602. In a particular embodiment, the depression 608 is sized and shaped to receive the articulation member 508 of the superior component 500. For example, the depression 608 can have a hemi-spherical shape. Alternatively, the depression 608 can have an elliptical shape, a cylindrical shape, or other arcuate shape. As further illustrated, the inferior component 600 can include a first conformable body 620, a second conformable body 609 and a third conformable body 611. In the embodiment shown, the second conformable body 609 and the third conformable body 611 overlie the first conformable body 620. All three of the conformable bodies can be configured and prepared as described supra.

FIG. 6 through FIG. 9 indicate that the inferior component 600 can include an inferior keel 648 that extends from inferior engagement surface 606. During installation, described below, the inferior keel 648 can at least partially engage a keel groove that can be established within a cortical rim of a vertebra, e.g., the keel groove 70 shown in FIG. 5.

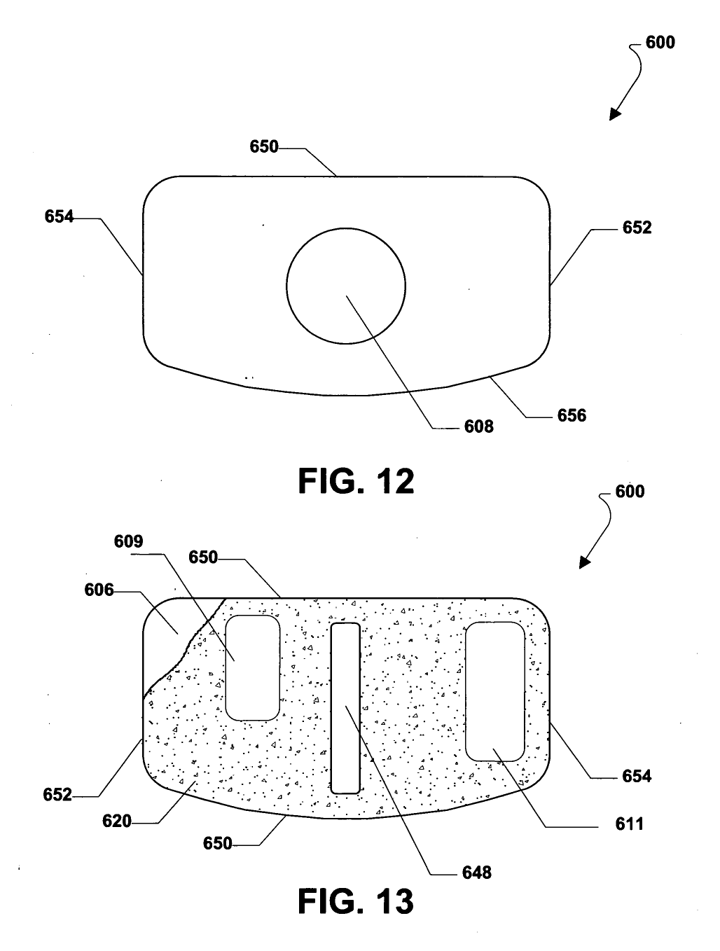

In a particular embodiment, as shown in FIG. 12 and FIG. 13, the inferior component 600 can be shaped to match the shape of the superior component 500, shown in FIG. 10 and FIG. 11. Further, the inferior component 600 can be generally rectangular in shape. For example, the inferior component 600 can have a substantially straight posterior side 650. A first straight lateral side 652 and a second substantially straight lateral side 654 can extend substantially perpendicular from the posterior side 650 to an anterior side 656. In a particular embodiment, the anterior side 656 can curve outward such that the inferior component 600 is wider through the middle than along the lateral sides 652, 654. Further, in a particular embodiment, the lateral sides 652, 654 are substantially the same length.

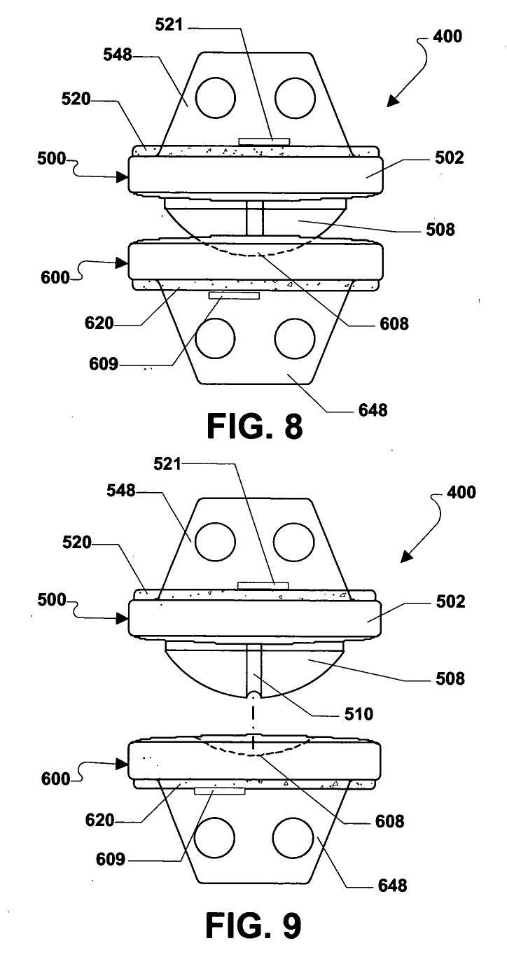

FIG. 6 and FIG. 8 show that the inferior component 600 includes a first implant inserter engagement hole 660 and a second implant inserter engagement hole 662. In a particular embodiment, the implant inserter engagement holes 660, 662 are configured to receive respective dowels, or pins, that extend from an implant inserter (not shown) that can be used to facilitate the proper installation of an intervertebral prosthetic implant, e.g., the intervertebral prosthetic implant 400 shown in FIG. 6 through FIG. 11.

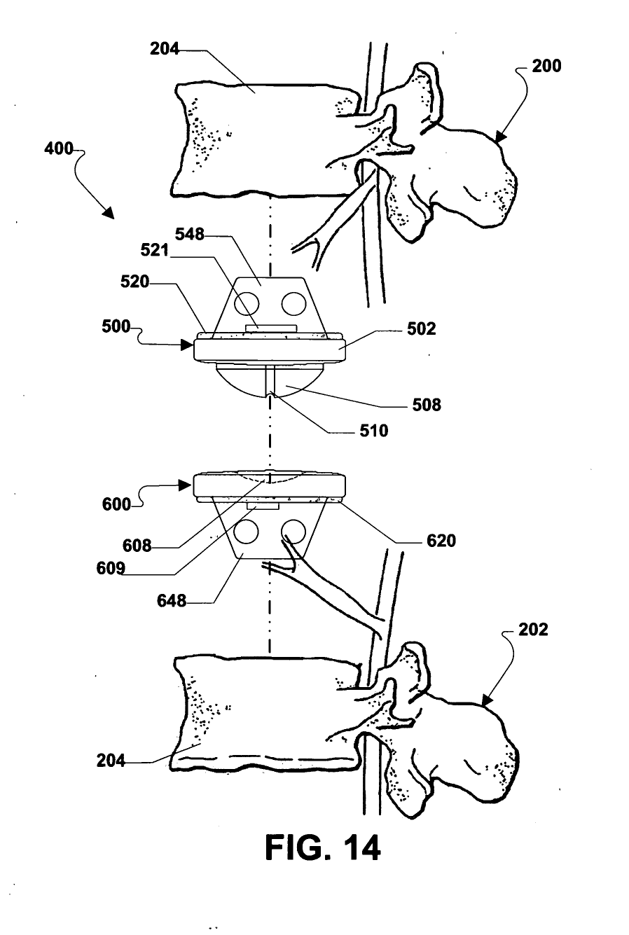

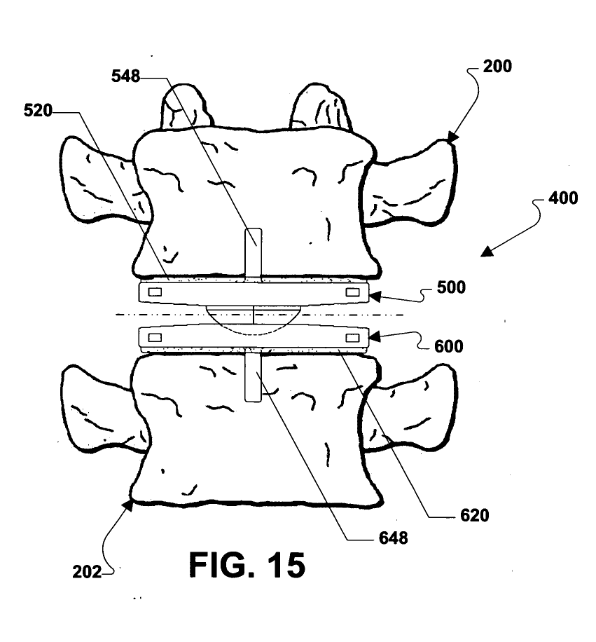

Referring to FIG. 14 and FIG. 15, an intervertebral prosthetic implant is shown between the superior vertebra 200 and the inferior vertebra 202, previously introduced and described in conjunction with FIG. 4. In a particular embodiment, the intervertebral prosthetic implant is the intervertebral prosthetic implant 400 described in conjunction with FIG. 6 through FIG. 13.

As shown in FIG. 14 and FIG. 15, the intervertebral prosthetic implant 400 is installed within the intervertebral space 214 that can be established between the superior vertebra 200 and the inferior vertebra 202 by removing vertebral disc material (not shown). In a particular embodiment, the superior keel 548 of the superior component 500 can at least partially engage the cancellous bone and cortical rim of the superior vertebra 200. Also, in a particular embodiment, the inferior keel 648 of the inferior component 600 can at least partially engage the cancellous bone and cortical rim of the inferior vertebra 202.

FIG. 15 indicates that the conformable bodies can engage the superior vertebra 200, e.g., the cortical rim and cancellous bone of the superior vertebra 200. The conformable bodies can mold, or otherwise form, to match the uneven or irregular shape of the cortical rim and cancellous bone of the superior vertebra 200. In a particular embodiment, the conformable bodies can increase the contact area between the superior vertebra 200 and the superior support plate 502. As such, the superior the conformable bodies can substantially reduce the contact stress between the superior vertebra 200 and the superior support plate 502. The conformable bodies on the inferior support plate can function in a similar manner.

As illustrated in FIG. 14 and FIG. 15, the articulation member 508 that extends from the superior component 500 of the intervertebral prosthetic implant 400 can at least partially engage the depression 608 that is formed within the inferior component 600 of the intervertebral prosthetic implant 400. It is to be appreciated that when the intervertebral prosthetic implant 400 is installed between the superior vertebra 200 and the inferior vertebra 202, the intervertebral prosthetic implant 400 allows relative motion between the superior vertebra 200 and the inferior vertebra 202. Specifically, the configuration of the superior component 500 and the inferior component 600 allows the superior component 500 to rotate with respect to the inferior component 600. As such, the superior vertebra 200 can rotate with respect to the inferior vertebra 202.

In a particular embodiment, the intervertebral prosthetic implant 400 can allow angular movement in any radial direction relative to the intervertebral prosthetic implant 400. Further, as depicted in FIG. 15, the inferior component 600 can be placed on the inferior vertebra 202 so that the center of rotation of the inferior component 600 is substantially aligned with the center of rotation of the inferior vertebra 202. Similarly, the superior component 500 can be placed relative to the superior vertebra 200 so that the center of rotation of the superior component 500 is substantially aligned with the center of rotation of the superior vertebra 200. Accordingly, when the vertebral disc, between the inferior vertebra 202 and the superior vertebra 200, is removed and replaced with the intervertebral prosthetic implant 400 the relative motion of the vertebrae 200, 202 provided by the vertebral disc is substantially replicated.

It will be understood that each of the elements described above, or two or more together, may also find utility in applications differing from the types described herein. While the invention has been illustrated and described as embodied in a conformable orthopedic implant, it is not intended to be limited to the details shown, since various modifications and substitutions can be made without departing in any way from the spirit of the present invention. For example, although many examples of various alternative biocompatible chemicals and materials have been presented throughout this specification, the omission of a possible item is not intended to specifically exclude its use in or in connection with the claimed invention. As such, further modifications and equivalents of the invention herein disclosed may occur to persons skilled in the art using no more than routine experimentation, and all such modifications and equivalents are believed to be within the spirit and scope of the invention as defined by the following claims.

1. A compound orthopedic implant, comprising
a first conformable body and
a second conformable body overlying the first conformable body.

2. (canceled)

3. The compound orthopedic implant of claim 1, wherein the second conformable body completely overlies the first conformable body.

4. The compound orthopedic implant of claim 1, wherein the first conformable body comprises collagen

5. The compound orthopedic implant of claim 4, wherein the first conformable body further comprises ceramic particles.

6. (canceled)

7. The compound orthopedic implant of claim 5, wherein the ceramic particles comprise hydroxyapatite particles.

8-9. (canceled)

10. The compound orthopedic implant of claim 5, wherein the second conformable body comprises collagen.

11. The compound orthopedic implant of claim 10, wherein the second conformable body further comprises ceramic particles.

12-15. (canceled)

16. The compound orthopedic implant of claim 1, wherein at least one of the first conformable body or the second conformable body comprises a therapeutic agent.

17. The compound orthopedic implant of claim 16, wherein the first conformable body comprises a therapeutic agent and the second conformable body comprises a therapeutic agent.

18. The compound orthopedic implant of claim 16, wherein the first conformable body comprises a therapeutic agent and the second conformable body is substantially free of therapeutic agents.

19. (canceled)

20. The compound orthopedic implant of claim 16, wherein the therapeutic agent includes a bone morphogenetic protein.

21. The compound orthopedic implant of claim 11, wherein the weight ratio of the ceramic particles to the collagen in the first conformable body is between about 5:1 and about 20:1.

22. The compound orthopedic implant of claim 21, wherein the weight ratio of the ceramic particles to the collagen in the second conformable body is between about 5:1 and about 20:1.

23. The compound orthopedic implant of claim 21, wherein the weight ratio of the ceramic particles to the collagen in the second conformable body is greater than the weight ratio of the ceramic particles to the collagen in the first conformable body.

24. The compound orthopedic implant of claim 24, wherein the weight ratio of the ceramic particles to the collagen in the second conformable body is between about 22:1 and 40:1.

25. The compound orthopedic implant of claim 11, wherein extent of crosslinking of the first conformable body is less than the extent of crosslinking of the second conformable body.

26. The compound orthopedic implant of claim 1, further comprising a third conformable body overlying the first conformable body.

27. The compound orthopedic implant of claim 26, wherein the second conformable body overlies a first portion of the first conformable body and the third conformable body overlies a second portion of the first conformable body.

28-29. (canceled)

30. The compound orthopedic implant of claim 1, further comprising a third conformable body overlying the second conformable body.

31. (canceled)

32. A compound orthopedic implant comprising
a first conformable body comprising ceramic particles and collagen and
a second conformable body overlying the first conformable body, the second conformable body comprising ceramic particles and collagen, wherein at least one of the first conformable body or the second conformable body comprises a therapeutic agent.

33-35. (canceled)

36. A kit for field use, the kit comprising:
a first conformable body;
a second conformable body; and
instructions for utilizing the first and second comfortable bodies as a compound orthopedic implant.

37-45. (canceled)
